(19) United States
(12) Patent Application Publication
(10) Pub. No.: US 2014/0162294 A1
(43) Pub. Date: Jun. 12, 2014

(54) METHODS AND COMPOSITIONS FOR ASSAYING VITAMIN D

(71) Applicant: GENERAL ATOMICS, San Diego, CA (US)

(72) Inventors: Chong-Sheng Yuan, San Diego, CA (US); Xiaoru Chen, San Diego, CA (US); Ruffy Baldemeca, San Diego, CA (US)

(73) Assignee: General Atomics, San Diego, CA (US)

(21) Appl. No.: 13/707,514

(22) Filed: Dec. 6, 2012

(51) Int. Cl. G01N 33/82

(52) U.S. Cl. CPC G01N 33/82
USPC 435/7.92; 436/501

(57) ABSTRACT

The present invention provides methods for assaying a vitamin D moiety in a sample, using a water miscible organic solvent, a specific binding partner that specifically binds to said vitamin D moiety, the binding partner being different from a natural vitamin D binding protein for the vitamin D moiety, and a water soluble polymer that facilitates binding between the specific binding partner and the vitamin D moiety. Kits and reaction mixtures for assaying a vitamin D moiety in a sample are also provided.

### FIELD OF THE INVENTION

This invention generally relates to the field of vitamin D detection. In particular, the invention provides novel methods and kits for assaying a vitamin D moiety in a sample such as a biological fluid.

### BACKGROUND OF THE INVENTION

Vitamin D is a steroid-like, fat soluble prohormone. Vitamin D has two main forms: D2 (ergocalciferol) and D3 (cholecalciferol). Vitamin D3 can be manufactured by the body upon exposure to UV radiation. Both Vitamin D3 and Vitamin D2 are converted to the active hormone 1,25-dihydroxy Vitamin D through their metabolism in the liver and kidney.

Vitamin D3 is synthesized in skin by exposure to sunlight (ultraviolet radiation) and obtained from the diet primarily from fish liver oils and egg yolks. Vitamin D2 is obtained mainly from nutritional supplements and the only prescription drug for Vitamin D deficiency is made of Vitamin D2. Vitamin D3 or D2 is metabolized by the liver to 25(OH)D, which is then converted by the kidneys to 1,25(OH)2D. 25(OH) Vitamin D is the major circulating form which reflects the levels of Vitamin D in the body, but 1,25(OH)2 Vitamin D is the most biologically active form.

inadequate exposure to sunlight or low intake from diet or supplements may cause vitamin D deficiency. Vitamin D deficiency impairs bone mineralization, causing rickets in children and osteomalacia in adults and may contribute to osteoporosis. Recent studies have shown that Vitamin D deficiency is also linked to cancers, cardiovascular diseases, diabetes, multiple sclerosis, Parkinson disease, Alzheimer's disease, drug efficacy, and all-cause mortality.

A typical normal or sufficient range for Vitamin D is about 30-100 ng/mL. Vitamin D level at about 10-30 ng/mL is considered deficient. Vitamin D level less than 10 ng/mL is considered severely deficient. Vitamin D level more than 150 ng/mL is considered toxic.

Various vitamin D assays are known in the art. For example, Various vitamin D assays are disclosed in U.S. Pat. Nos. 5,821,020, 7,087,395 B1, 7,482,162 B2, 7,964,363 B2, 8,133,694 B2, U.S. patent publication No. 2004/0132104 A1 and WO 2012/091569 A1.

There remains a need for a reliable, sensitive and specific method for assaying a vitamin D moiety in a sample such as a biological fluid, particularly one that can be conducted as a homogeneous assay and/or is amenable to automated clinical chemistry analyzers in the typical clinical laboratory settings.

### BRIEF SUMMARY OF THE INVENTION

In one aspect, the present invention provides methods for assaying a vitamin D moiety in a sample, which method comprise: a) contacting a sample containing or suspected of containing a vitamin D moiety with a water miscible organic solvent, a specific binding partner that specifically binds to said vitamin D moiety, if present in said sample, said binding partner being different from a natural vitamin D binding protein for said vitamin D moiety, and a water soluble polymer that facilitates binding between said specific binding partner and said vitamin D moiety; and b) assessing binding between said specific binding partner and said vitamin D moiety to determine the presence, absence and/or amount of said vitamin D moiety in said sample.

In another aspect, the present invention provides kits for assaying a vitamin D moiety in a sample, which kits comprise: a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

In still another aspect, the present invention provides reaction mixtures for assaying a vitamin D moiety in a sample, which reaction mixtures comprise: a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

### BRIEF DESCRIPTION OF THE DRAWINGS

FIG. 1 illustrates an exemplary 25(OH)D immunoturbidimetric (three reagent on Hitachi 917) calibration curve.

FIG. 2 illustrates an exemplary total 25(OH)D method comparison (three reagent immunoturbidimetric vs. EIA).

FIG. 3 illustrates an exemplary 25(OH)D immunoturbidimetric (two reagent on modular P) calibration curve.

FIG. 4 illustrates an exemplary 25(OH)D immunoturbidimetric (two reagent on modular P) linearity.

FIG. 5 illustrates an exemplary total 25(OH)D method comparison (two reagent immunoturbidimetric vs. LC-MS).

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “vitamin D moiety” refers to all members or forms of the Vitamin D family which is a group of fat-soluble secosteroids responsible for intestinal absorption of calcium and phosphate. Exemplary vitamin D forms include vitamin D₁, (ergocalciferol), D₃ (cholecalciferol), D₄, and D₅. Exemplary vitamin D moieties also include calcidiol, which is also known as calcifediol (INN), 25-hydroxycholecalciferol, or 25-hydroxyvitamin D—abbreviated 25(OH)D; and which is the specific vitamin D metabolite that is measured in serum to determine a person's vitamin D status, and calcitriol, the biologically active form of vitamin D.

As used herein, a “binding partner (or binder)” refers to any substance that binds to a target or an analyte, e.g., a vitamin D moiety, with desired affinity and/or specificity. Non-limiting examples of the binding reagent include cells, cellular organelles, viruses, particles, microparticles, molecules, or an aggregate or complex thereof, or an aggregate or complex of molecules.

As used herein, “antibody” includes not only intact polyclonal or monoclonal antibodies, but also fragments thereof (such as Fab, Fab′, F(ab′)₂, Fv), single chain (ScFv), a diabody, a multi-specific antibody formed from antibody fragments, mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class.

As used herein, the term “specifically binds” refers to the specificity of a binding reagent, e.g., an antibody, such that it preferentially binds to a defined analyte or target e.g., a vitamin D moiety. Recognition by a binding reagent or an antibody of a particular analyte or target in the presence of other potential targets is one characteristic of such binding. In some embodiments, a binding reagent that specifically binds to an analyte avoids binding to other interfering moiety or moieties in the sample to be tested.

As used herein the term “avoids binding” refers to the specificity of particular binding reagents, e.g., antibodies or antibody fragments. Binding reagents, antibodies or antibody fragments that avoid binding to a particular moiety generally contain a specificity such that a large percentage of the particular moiety would not be bound by such binding reagents, antibodies or antibody fragments. This percentage generally lies within the acceptable cross reactivity percentage with interfering moieties of assays utilizing the binding reagents or antibodies directed to detecting a specific target. Frequently, the binding reagents, antibodies or antibody fragments of the present disclosure avoid binding greater than about 90% of an interfering moiety, although higher percentages are clearly contemplated and preferred. For example, binding reagents, antibodies or antibody fragments of the present disclosure avoid binding about 91%, about 92%, about 93%, about 94%, about 95%, about 96%, about 97%, about 98%, about 99%, and about 99% or more of an interfering moiety. Less occasionally, binding reagents, antibodies or antibody fragments of the present disclosure avoid binding greater than about 50%, greater than about 60%, greater than about 70%, or greater than about 75%, or greater than about 80%, or greater than about 85% of an interfering moiety.

A “vitamin D binding protein” is also known as gc-globulin (group-specific component). As used herein, a “vitamin D binding protein” refers to a Vitamin D-binding protein in the albumin family. A “vitamin D binding protein” is often found in plasma, ascitic fluid, cerebrospinal fluid and/or on the surface of many cell types. A “vitamin D binding protein” often binds to vitamin D and its metabolites and transports them to target tissues in vivo. An exemplary “vitamin D binding protein” in humans is encoded by the GC gene.

As used herein the term “assessing” is intended to include quantitative and qualitative determination in the sense of obtaining an absolute value for the amount or concentration of the analyte present in the sample, and also of obtaining an index, ratio, percentage, visual or other value indicative of the level of analyte in the sample. Assessment may be direct or indirect and the chemical species actually detected need not of course be the analyte itself but may for example be a derivative thereof or some further substance.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “blood sample” refers to a whole blood sample or a plasma or serum fraction derived therefrom. Preferably, the blood sample refers to a human blood sample such as whole blood or a plasma or serum fraction derived therefrom. Also preferably, the blood sample is pre-treated before the assay by removing substantially all hemoglobin (i.e., red blood cells) in order to eliminate or significantly reduce the oxidative interference from the hemoglobin molecules.

As used herein the term “whole blood” refers to a blood sample that has not been fractionated and contains both cellular and fluid components. As used herein, “whole blood” refers to freshly drawn blood which is tested before it clots, or a conventionally-drawn blood sample, which may be drawn into a vacutainer, and which may contain an anticoagulant, such as lithium-heparin, EDTA etc., or to which one or more other standard clinical agents may be added in the course of routine clinical testing.

As used herein, the phrase “substantially all hemoglobin has been removed” refers to a blood sample wherein preferably at least about 50%, 60% or 70%, more preferably, at least about 80%, 90% or 95%, and most preferably, at least about 96%, 97%, 98%, 99 or 100% of all hemoglobin-containing red blood cells in the sample have been removed to eliminate or significantly reduce the oxidative interference from hemoglobin.

As used herein, the term “plasma” refers to the fluid, non-cellular component of the whole blood. Depending on the separation method used, plasma may be completely free of cellular components, or may contain various amounts of platelets and/or a small amount of other cellular components. Because plasma includes various clotting factors such as fibrinogen, the term “plasma” is distinguished from “serum” as set forth below.

As used herein, the term “serum” refers to whole mammalian serum, such as whole human serum. Further, as used herein, “serum” refers to blood plasma from which clotting factors (e.g., fibrinogen) have been removed.

As used herein, the term “fluid” refers to any composition that can flow. Fluids thus encompass compositions that are in the form of semi-solids, pastes, solutions, aqueous mixtures, gels, lotions, creams and other such compositions.

As used herein, the term “disease” or “disorder” refers to a pathological condition in an organism resulting from, e.g., infection or genetic defect, and characterized by identifiable symptoms.

As used herein, “contacting” means bringing two or more components together. “Contacting” can be achieved by mixing all the components in a fluid or semi-fluid mixture. “Contacting” can also be achieved when one or more components are brought into contact with one or more other components on a solid surface such as a solid tissue section or a substrate.

As used herein, the term “comparing” generally means examining in order to note similarities or differences between two or more values. Preferably, “comparing” refers to quantitative comparisons such as, for example, subtracting one value from another, calculating a ratio of two values, calculating a percentage of one value with respect to another, or combining these types of calculations to produce a single number. As used herein, “comparing” further refers to comparisons made by a human, comparisons made by a computer or other processor, and comparisons made by a human in combination with a computer or other processor.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### B. METHODS FOR ASSAYING A VITAMIN D MOIETY

In one aspect, the present invention provides methods for assaying a vitamin D moiety in a sample, which method comprises: a) contacting a sample containing or suspected of containing a vitamin D moiety with a water miscible organic solvent, a specific binding partner that specifically binds to said vitamin D moiety, if present in said sample, said binding partner being different from a natural vitamin D binding protein for said vitamin D moiety, and a water soluble polymer that facilitates binding between said specific binding partner and said vitamin D moiety; and b) assessing binding between said specific binding partner and said vitamin D moiety to determine the presence, absence and/or amount of said vitamin D moiety in said sample.

In some embodiments, the present methods do not comprise a step of removing protein from the sample (See e.g., U.S. Pat. No. 5,821,020), such as the natural vitamin D binding protein for the vitamin D moiety, prior to assessing binding between the specific binding partner and the vitamin D moiety. In other embodiments, the present methods do not comprise any wash step.

The present methods can be conducted in any suitable assay format. In some embodiments, the present methods are conducted as a homogeneous assay. In other embodiments, the present methods are conducted as a heterogeneous assay.

A sample can be contacted with a water miscible organic solvent, a specific binding partner and a water soluble polymer in any suitable manner or order. For example, a sample can be contacted with a water miscible organic solvent, a specific binding partner and a water soluble polymer in a single step, or in multiple steps, e.g., 2 or 3 steps. The exemplary contact order can be: 1) a water miscible organic solvent, a specific binding partner and a water soluble polymer; 2) a water miscible organic solvent, a water soluble polymer and a specific binding partner; 3) a specific binding partner, a water miscible organic solvent and a water soluble polymer; 4) a specific binding partner, a water soluble polymer and a water miscible organic solvent; 5) a water soluble polymer, a water miscible organic solvent and a specific binding partner; and 6) a water soluble polymer, a specific binding partner and a water miscible organic solvent.

In another example, a sample can be contacted with two substances and then be contacted with the third substance. The exemplary contact order can be: 1) contact with a water miscible organic solvent and a specific binding partner together and followed by contact with a water soluble polymer; 2) contact with a water miscible organic solvent and a water soluble polymer and followed by a specific binding partner; and 3) contact with a specific binding partner and a water soluble polymer together and followed by contact with a water miscible organic solvent. In some embodiments, a sample is contacted with a single reaction mixture comprising the water miscible organic solvent, the specific binding partner and the water soluble polymer.

In some embodiments, the present methods do not comprise a step of contacting the sample with 8-anilino-1-napthalenesulfonic acid ammonium salt and/or 3-(acetonylbenzyl)-4-hydroxycoumarin. See e.g., U.S. Pat. No. 7,482,162 B2. In other embodiments, the present methods do not comprise a step of contacting the sample with a non-competitive displacement agent that separates the vitamin D moiety from its binding protein in the sample. Id.

The pH in a final reaction mixture comprising the sample, the water miscible organic solvent, the specific binding partner and the water soluble polymer can be at any suitable value or range. In some embodiments, the pH in a final reaction mixture comprising the sample, the water miscible organic solvent, the specific binding partner and the water soluble polymer is at 4 or higher. In other embodiments, the pH in a final reaction mixture comprising the sample, the water miscible organic solvent, the specific binding partner and the water soluble polymer is at 13 or lower. In still other embodiments, the pH in a final reaction mixture comprising the sample, the water miscible organic solvent, the specific binding partner and the water soluble polymer is in a range from about 4 to about 13, e.g., at about 4, 4.5, 5, 5.5, 5.6, 5.7, 5.8, 5.9, 6, 6.5, 7, 7.5, 8, 8.5, 9, 9.5, 10, 10.5, 11, 11.5, 12, 12.5, or 13.

In some embodiments, the present methods do not comprise a step of contacting the sample with a vitamin D releasing composition comprising a cyclodextrin, a sodium salicylate and NaOH, such as the vitamin D releasing composition disclosed in U.S. Pat. No. 7,087,395 B1.

In some embodiments, the present methods do not comprise a step of contacting the sample with a perfluoro alkyl acid, or a salt thereof, to release 25(OH) vitamin D from vitamin D binding protein, such as the step disclosed in WO 2012/091569 A1.

In some embodiments, the present methods do not comprise a step of contacting the sample with a serine protease with endo- and exoproteolytic activity to digest vitamin D binding proteins in the sample, such as the step disclosed in U.S. Pat. No. 7,964,363 B2.

The present methods can be used for any suitable purpose. In some embodiments, the present methods can be used to assess status of the vitamin D moiety in a subject, and the sample is a biological sample obtained and/or derived from the subject. The present methods can be used for assess status of the vitamin D moiety in any suitable subject, e.g., a mammal, a non-human mammal, a human or an experimental animal.

The present methods can be used for assaying a vitamin D moiety in any suitable sample. In some embodiments, the sample is a biological fluid, e.g., whole blood, plasma, serum or urine.

The present methods can be used for assaying any suitable vitamin D moiety in a sample. In some embodiments, the vitamin D moiety is vitamin D₃, vitamin D₂, a vitamin D metabolite or 1,25-dihydroxyvitamin D_{3 [}1,25(OH)₂D₃]. In other embodiments, the vitamin D moiety is 25-hydroxy-vitamin D (25(OH)D), e.g., 25(OH)D3, 25(OH)D2 or a sum of 25(OH)D2 and 25(OH)D3.

Any suitable water miscible organic solvent can be used in the present methods. In some embodiments, the water miscible organic solvent can be dimethyl sulfoxide, dimethyl formamide, acetonitrile, methanol, ethanol, isopropyl alcohol, dimethyl acetamide, and a combination thereof.

The water miscible organic solvent can be used at any suitable concentration. In some embodiments, the concentration of the water miscible organic solvent(s) in the final reaction mixture ranges from about 0.25% (V/V) to about 20% (V/V), e.g., about 0.25% (V/V), 0.5% (V/V), 0.75% (V/V), 1% (V/V), 2% (V/V), 3% (V/V), 4% (V/V), 5% (V/V), 6% (V/V), 7% (V/V), 8% (V/V), 9% (V/V), 10% (V/V), 11% (V/V), 12% (V/V), 13% (V/V), 14% (V/V), 15% (V/V), 16% (V/V), 17% (V/V), 18% (V/V), 19% (V/V), or 20% (V/V).

Any suitable specific binding partner that specifically binds to the vitamin D moiety can be used in the present methods. In some embodiments, the specific binding partner that specifically binds to the vitamin D moiety is an antibody that specifically binds to the vitamin D moiety. In other embodiments, the antibody specifically binds to 25(OH)D. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

Any suitable water soluble polymer can be used in the present methods. In some embodiments, a synthetic water soluble polymer and/or a natural water soluble polymer can be used. Any suitable synthetic water soluble polymer can be used in the present methods. Exemplary synthetic water soluble polymers include a polyoxyethylene (POE) (other names of the water soluble polymer being polyethylene glycol (PEG) or polyethylene oxide (PEO)), a polyvinyl pyrrolidone (PVP), a polyvinyl alcohol (PVA), a polyacrylic acid (PAA), a polyacrylamide, a poly-N-(2-hydroxypropyl)methacrylamide (HPMA), a poly-divinyl ether-maleic anhydride (DIVEMA), a polyoxazoline, a polyphosphate, a polyphosphazene, a polyethyleneimine, a polyglycosylethyl methacrylate, and a combination thereof. Exemplary natural water soluble polymers include a chitosan derivative, a dextran, a carrageenan, a cellulose ether, a hyaluronic acid (HA), and a starch or starch based derivative, and a combination thereof. In other embodiments, water soluble polymers disclosed in U.S. Pat. No. 6,548,310 B1 and Kadajji and Betageri, Polymers, 3: 1972-2009 (2011) can be used.

In some embodiments, the water soluble polymer is a non-ionic water soluble polymer, e.g., polyoxyethylene, polyvinyl pyrrolidone (PVP), a polyvinyl alcohol (PVA), polyacrylamide, poly-N-(2-hydroxypropyl)methacrylamide (HPMA).

The water soluble polymer can have any suitable size or molecular weight. For example, the water soluble polymer can have a molecular weight from about 1,000 to about 2,000,000, e.g., 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,500,000, or 2,000,000. In some embodiments, the water soluble polymer can have a molecular weight from about 2,000 to about 1,000,000.

In some embodiments, the water soluble polymer is polyvinyl pyrrolidone, polyethyleneimine, polyoxyethylene, or polyglycosylethyl methacrylate.

In some embodiments, the water soluble polymer is polyoxyethylene with a molecular weight from about 3,000 to about 500,000. For example, the polyoxyethylene can have a molecular weight of about 3,000, 4,000, 5,000, 6,000, 7,000, 8,000, 9,000, 10,000, 50,000, 100,000, 150,000, 200,000, 250,000, 300,000, 350,000, 400,000, 450,000, or 500,000.

In some embodiments, the water soluble polymer is polyvinyl pyrrolidone with a molecular weight from about 20,000 to about 750,000. For example, the polyvinyl pyrrolidone can have a molecular weight of about 20,000, 30,000, 40,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000 or 750,000.

In some embodiments, the water soluble polymer is dextran with a molecular weight from about 5,000 to about 700,000. For example, the dextran can have a molecular weight of about 5,000, 6,000, 7,000, 8,000, 9,000, 10,000, 50,000, 100,000, 150,000, 200,000, 250,000, 300,000, 350,000, 400,000, 450,000, 500,000, 550,000, 600,000, 650,000 or 700,000.

The water soluble polymer can be used at any suitable level or concentration. In some embodiments, the concentration of the water soluble polymer in the final reaction mixture ranges from about 0.025% (W/V) to about 10% (W/V), e.g., 0.025% (W/V), 0.05% (W/V), 0.075% (W/V), 0.1% (W/V), 0.2% (W/V), 0.3% (W/V), 0.4% (W/V), 0.5% (W/V), 0.6% (W/V), 0.7% (W/V), 0.8% (W/V), 0.9% (W/V), 1% (W/V), 2% (W/V), 3% (W/V), 4% (W/V), 5% (W/V), 6% (W/V), 7% (W/V), 8% (W/V), 9% (W/V), or 10% (W/V).

The present methods can be used in any suitable assay format. In some embodiments, the present methods are conducted using a particle-enhanced immunoturbidimetric method. See e.g., U.S. Pat. No. 4,703,018, In other embodiments, the present methods are conducted using a particle-enhanced immunonephelometric method. See e.g., U.S. Pat. No. 4,690,906. In still other embodiments, the present methods are conducted using a magnetic particle (bead) based immunoassay method. See e.g., Yu et al., Journal of immunological Methods, 218(1-2):1-8 (1998). In yet other embodiments, the present methods are conducted using a particle based immunoagglutination assay method. See e.g., Wang et al., Clinical Chemistry, 52(11):2065-2071 (2006).

Any suitable particle can be used in the present particle based assay formats. In some embodiments, the particle comprises polystyrene, polymethyl methacrylate, polymethyl naphthalene, poly(divinylbenzene), polyvinyl naphthalene, co-polymer of styrene, acrylic acid divinylbenzene, naphathalene, carbon 60, magnetic beads, gold, silver, silica, silicon dioxide, chromium dioxide, and/or titanium dioxide. In other embodiments, the particle is a nanoparticle. The nanoparticle can have any suitable size or diameter. For example, the nanoparticle can have any diameter ranging from about 30 nm to about 500 nm, e.g., about 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm.

In some embodiments, the present methods are conducted using a homogeneous assay format. In other embodiments, the present methods are conducted using a heterogeneous assay format. In still other embodiments, the present methods are conducted using a sandwich or competitive assay format. In yet other embodiments, the present methods are conducted using a format of an enzyme-linked immunosorbent assay (ELISA), cloned enzyme donor immunoassay (CEDIA), enzyme multiplied immunoassay technique (EMIT), immunoblotting, immunoprecipitation, radioimmunoassay (RIA), immunostaining, latex agglutination, indirect hemagglutination assay (IHA), complement fixation, indirect immunofluorescent assay (IFA), nephelometry, chemiluminescence assay, lateral flow immunoassay, immunoinhibition assay or immune-avidity assay.

Any suitable homogenous assay format can be used. In some embodiments, a homogenous assay is conducted in a single reaction mixture without phase separation or washing step, such as the particle separation and washing steps disclosed in U.S. patent publication No. U.S. 2004/0132104 A1 or U.S. Pat. No. 8,133,694 B2.

The present methods can be conducted in any suitable time. In some embodiments, the present methods have a total assay time that is at about 30 minutes or shorter, e.g., about 29, 28, 27, 26, 25, 24, 23, 22, 21, 20, 19, 18, 17, 16, 15, 14, 13, 12, 11, 10, 9, 8, 7, 6, 5, 4, 3 minutes.

The present methods can be conducted on any suitable analytic instruments. In some embodiments, the present methods are conducted on a general chemistry analyzer or a clinical chemistry analyzer, e.g., general chemistry analyzer or clinical chemistry analyzer from Roche, Hitachi, Modular P, Cobas series, Beckman/Olympus AU series, Beckman Synchron and DXC series, or Abbot Architect series.

### C. KITS FOR ASSAYING A VITAMIN D MOIETY AND USES THEREOF

In another aspect, the present invention provides a kit for assaying a vitamin D moiety in a sample, which kit comprises: a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

Any suitable water miscible organic solvent can be used in the present kits. In some embodiments, the water miscible organic solvent can be dimethyl sulfoxide, dimethyl formamide, acetonitrile, methanol, ethanol, isopropyl alcohol, dimethyl acetamide, and a combination thereof.

The water miscible organic solvent can be used at suitable concentration. In some embodiments, the concentration of the water miscible organic solvent(s) in the final reaction mixture ranges from about 0.25% (V/V) to about 20% (V/V), e.g., about 0.25% (V/V), 0.5% (V/V), 0.75% (V/V), 1% (V/V), 2% (V/V), 3% (V/V), 4% (V/V), 5% (V/V), 6% (V/V), 7% (V/V), 8% (V/V), 9% (V/V), 10% (V/V), 11% (V/V), 12% (V/V), 13% (V/V), 14% (V/V), 15% (V/V), 16% (V/V), 17% (V/V), 18% (V/V), 19% (V/V), or 20% (V/V).

Any suitable specific binding partner that specifically binds to the vitamin D moiety can be used in the present kits. In some embodiments, the specific binding partner that specifically binds to the vitamin D moiety is an antibody that specifically binds to the vitamin D moiety. In other embodiments, the antibody specifically binds to 25(OH)D. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

Any suitable water soluble polymer can be used in the present kits. In some embodiments, a synthetic water soluble polymer and/or a natural water soluble polymer can be used. Any suitable synthetic water soluble polymer can be used in the present kits. Exemplary synthetic water soluble polymers include a polyoxyethylene (POE) (other names of the water soluble polymer being polyethylene glycol (PEG) or polyethylene oxide (PEO)), a polyvinyl pyrrolidone (PVP), a polyvinyl alcohol (PVA), a polyacrylic acid (PAA), a polyacrylamide, a poly-N-(2-hydroxypropyl)methacrylamide (HPMA), a poly-divinyl ether-maleic anhydride (DIVEMA), a polyoxazoline, a polyphosphate, a polyphosphazene, a polyethyleneimine, a polyglycosylethyl methacrylate, and a combination thereof. Exemplary natural water soluble polymers include a chitosan derivative, a dextran, a carrageenan, a cellulose ether, a hyaluronic acid (HA), and a starch or starch based derivative, and a combination thereof. In other embodiments, water soluble polymers disclosed in U.S. Pat. No. 6,548,310 B1 and Kadajji and Betageri, Polymers, 3: 1972-2009 (2011) can be used.

In some embodiments, the water soluble polymer is a non-ionic water soluble polymer, e.g., polyoxyethylene, polyvinyl pyrrolidone (PVP), a polyvinyl alcohol (PVA), polyacrylamide, poly-N-(2-hydroxypropyl)methacrylamide (HPMA).

The water soluble polymer can have any suitable size or molecular weight. For example, the water soluble polymer can have a molecular weight of from about 1,000 to about 2,000,000, e.g., 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,500,000, or 2,000,000. In some embodiments, the water soluble polymer can have a molecular weight from about 2,000 to about 1,000,000.

In some embodiments, the water soluble polymer is polyvinyl pyrrolidone, polyethyleneimine, polyoxyethylene, or polyglycosylethyl methacrylate.

In some embodiments, the water soluble polymer is polyoxyethylene with a molecular weight of about 3,000 to about 500,000. For example, the polyoxyethylene can have a molecular weight of about 3,000, 4,000, 5,000, 6,000, 7,000, 8,000, 9,000, 10,000, 50,000, 100,000, 150,000, 200,000, 250,000, 300,000, 350,000, 400,000, 450,000, or 500,000.

In some embodiments, the water soluble polymer is polyvinyl pyrrolidone with a molecular weight of about 20,000 to about 750,000. For example, the polyvinyl pyrrolidone can have a molecular weight of about 20,000, 30,000, 40,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000 or 750,000.

In some embodiments, the water soluble polymer is dextran with a molecular weight of about 5,000 to about 700,000. For example, the dextran can have a molecular weight of about 5,000, 6,000, 7,000, 8,000, 9,000, 10,000, 50,000, 100,000, 150,000, 200,000, 250,000, 300,000, 350,000, 400,000, 450,000, 500,000, 550,000, 600,000, 650,000 or 700,000.

The water soluble polymer can be used at any suitable level or concentration. In some embodiments, the concentration of the water soluble polymer in the final reaction mixture ranges from about 0.025% (W/V) to about 10% (W/V), e.g., 0.025% (W/V), 0.05% (W/V), 0.075% (W/V), 0.1% (W/V), 0.2% (W/V), 0.3% (W/V), 0.4% (W/V), 0.5% (W/V), 0.6% (W/V), 0.7% (W/V), 0.8% (W/V), 0.9% (W/V), 1% (W/V), 2% (W/V), 3% (W/V), 4% (W/V), 5% (W/V), 6% (W/V), 7% (W/V), 8% (W/V), 9% (W/V), or 10% (W/V).

The present kits can comprise any additional suitable reagents or components. In some embodiments, the present kits further comprise means for assessing binding between the specific binding partner and the vitamin D moiety to determine the presence, absence and/or amount of the vitamin D moiety in the sample.

The reagents or components in the present kits can be formulated or arranged in any suitable fashion or form. In some embodiments, the present kits comprise the following reagents: (1) a first assay reagent comprising the water miscible organic solvent and the water soluble polymer; (2) a second assay reagent comprising a specific antibody against 25(OH)D; and (3) a third assay reagent comprising 25(OH)D coated particles.

The above kits can be used in a method for assaying a vitamin D moiety in a sample, which method comprises: a) forming a mixture of a sample, the first assay reagent and the second assay reagent and incubating the mixture for a period of time before adding the third assay reagent to the mixture; and b) quantifying the amount of 25(OH)D in the sample by measuring the optical change of the reaction mixture and using a set of 25(OH)D calibrators.

In other embodiments, the present kits comprise the following reagents: (1) a first assay reagent comprising the water miscible organic solvent, the water soluble polymer and an antibody against 25(OH)D; and (2) a second assay reagent comprising 25(OH)D coated particles.

The above kits can be used in a method for assaying a vitamin D moiety in a sample, which method comprises: a) forming a mixture of a sample and the first assay reagent and incubating the mixture for a period of time before adding the second assay reagent to the mixture; and b) quantifying the amount of 25(OH)D in the sample by measuring the optical change of the reaction mixture and using a set of 25(OH)D calibrators.

In still other embodiments, the present kits comprise (1) a first assay reagent comprising the water miscible organic solvent and the water soluble polymer, and (2) a solid surface comprising an immobilized substance that is the specific binding partner that specifically binds to a vitamin D moiety or its analog.

The above kits can be used in a method for assaying a vitamin D moiety in a sample, which method comprises: a) forming a mixture of a sample and the first assay reagent and incubating the mixture for a period of time; and b) contacting the mixture with the solid surface to determine the presence, absence and/or amount of the vitamin D moiety in the sample.

### D. REACTION MIXTURES FOR ASSAYING A VITAMIN D MOIETY IN A SAMPLE

In still another aspect, the present invention provides a reaction mixture for assaying a vitamin D moiety in a sample, which reaction mixture comprises: a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

Any suitable water miscible organic solvent can be used in the present reaction mixtures. In some embodiments, the water miscible organic solvent can be dimethyl sulfoxide, dimethyl formamide, acetonitrile, methanol, ethanol, isopropyl alcohol, dimethyl acetamide, and a combination thereof.

The water miscible organic solvent can be used at suitable concentration. In some embodiments, the concentration of the water miscible organic solvent(s) in the final reaction mixture ranges from about 0.25% (V/V) to about 20% (V/V), e.g., about 0.25% (V/V), 0.5% (V/V), 0.75% (V/V), 1% (V/V), 2% (V/V), 3% (V/V), 4% (V/V), 5% (V/V), 6% (V/V), 7% (V/V), 8% (V/V), 9% (V/V), 10% (V/V), 11% (V/V), 12% (V/V), 13% (V/V), 14% (V/V), 15% (V/V), 16% (V/V), 17% (V/V), 18% (V/V), 19% (V/V), or 20% (V/V).

Any suitable specific binding partner that specifically binds to the vitamin D moiety can be used in the present reaction mixtures. In some embodiments, the specific binding partner that specifically binds to the vitamin D moiety is an antibody that specifically binds to the vitamin D moiety. In other embodiments, the antibody specifically binds to 25(OH)D. Antibodies in any suitable forms can be used. For example, a polyclonal antibody or a monoclonal antibody can be used. In still other embodiments, exemplary antibodies disclosed in U.S. patent publication No. 2011/0097733 A1 can be used.

Any suitable water soluble polymer can be used in the present reaction mixtures. In some embodiments, a synthetic water soluble polymer and/or a natural water soluble polymer can be used. Any suitable synthetic water soluble polymer can be used in the present reaction mixtures. Exemplary synthetic water soluble polymers include a polyoxyethylene (POE) (other names of the water soluble polymer being polyethylene glycol (PEG) or polyethylene oxide (PEO)), a polyvinyl pyrrolidone (PVP), a polyvinyl alcohol (PVA), a polyacrylic acid (PAA), a polyacrylamide, a poly-N-(2-hydroxypropyl)methacrylamide (HPMA), a poly-divinyl ether-maleic anhydride (DIVEMA), a polyoxazoline, a polyphosphate, a polyphosphazene, a polyethyleneimine, a polyglycosylethyl methacrylate, and a combination thereof. Exemplary natural water soluble polymers include a chitosan derivative, a dextran, a carrageenan, a cellulose ether, a hyaluronic acid (HA), and a starch or starch based derivative, and a combination thereof. In other embodiments, water soluble polymers disclosed in U.S. Pat. No. 6,548,310 B1 and Kadajji and Betageri, Polymers, 3: 1972-2009 (2011) can be used.

In some embodiments, the water soluble polymer is a non-ionic water soluble polymer, e.g., polyoxyethylene, polyvinyl pyrrolidone (PVP), a polyvinyl alcohol (PVA), polyacrylamide, poly-N-(2-hydroxypropyl)methacrylamide (HPMA).

The water soluble polymer can have any suitable size or molecular weight. For example, the water soluble polymer can have a molecular weight of from about 1,000 to about 2,000,000, e.g., 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 1,500,000, or 2,000,000. In some embodiments, the water soluble polymer can have a molecular weight from about 2,000 to about 1,000,000.

In some embodiments, the water soluble polymer is polyvinyl pyrrolidone, polyethyleneimine, polyoxyethylene, or polyglycosylethyl methacrylate.

In some embodiments, the water soluble polymer is polyoxyethylene with a molecular weight of about 3,000 to about 500,000. For example, the polyoxyethylene can have a molecular weight of about 3,000, 4,000, 5,000, 6,000, 7,000, 8,000, 9,000, 10,000, 50,000, 100,000, 150,000, 200,000, 250,000, 300,000, 350,000, 400,000, 450,000, or 500,000.

In some embodiments, the water soluble polymer is polyvinyl pyrrolidone with a molecular weight of about 20,000 to about 750,000. For example, the polyvinyl pyrrolidone can have a molecular weight of about 20,000, 30,000, 40,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000 or 750,000.

In some embodiments, the water soluble polymer is dextran with a molecular weight of about 5,000 to about 700,000. For example, the dextran can have a molecular weight of about 5,000, 6,000, 7,000, 8,000, 9,000, 10,000, 50,000, 100,000, 150,000, 200,000, 250,000, 300,000, 350,000, 400,000, 450,000, 500,000, 550,000, 600,000, 650,000 or 700,000.

The water soluble polymer can be used at any suitable level or concentration. In some embodiments, the concentration of the water soluble polymer in the final reaction mixture ranges from about 0.025% (W/V) to about 10% (W/V), e.g., 0.025% (W/V), 0.05% (W/V), 0.075% (W/V), 0.1% (W/V), 0.2% (W/V), 0.3% (W/V), 0.4% (W/V), 0.5% (W/V), 0.6% (W/V), 0.7% (W/V), 0.8% (W/V), 0.9% (W/V), 1% (W/V), 2% (W/V), 3% (W/V), 4% (W/V), 5% (W/V), 6% (W/V), 7% (W/V), 8% (W/V), 9% (W/V), or 10% (W/V).

The present reaction mixtures can comprise any additional suitable reagents or components. In some embodiments, the reaction mixture further comprises a vitamin D moiety that is bound to the specific binding partner.

The present reaction mixtures can be formulated or arranged in any suitable fashion or manner. In some embodiments, the present reaction mixtures are contained in a single phase. In other embodiments, the present reaction mixtures are contained in multiple phases, e.g., two or three phases.

### E. EXEMPLARY EMBODIMENTS

In some embodiments, the present invention provides a method, e.g., a homogeneous or a heterogeneous method, for determining total 25-hydroxy-vitamin D [25(OH)D] concentrations in a sample, e.g., blood samples, wherein the dissociation of [25(OH)D] from vitamin D binding proteins (VBP), the binding of dissociated [25(OH)D] by a binding partner, e.g., an antibody, and the detection of [25(OH)D] in the sample is carried out in a single reaction mixture without phase separation or washing steps involved.

Of particular advantage of the embodiments is the significantly shorter total assay time (often <13 min) and its user friendly homogenous assay format that eliminates the needs for sample pre-treatment or phase separation/washing steps, and that allows the assay to be easily adapted for uses on general chemistry analyzers that are routinely used in clinical laboratories.

In some embodiments, the present invention provides a method of assaying a sample of blood or blood components for determining the amount of 25-hydroxy-vitamin D comprising: a) adding the sample to an aqueous mixture comprising a water miscible organic solvent, a binding partner specifically that binds [25(OH)D] that is different from vitamin D binding proteins in the sample, and a water soluble polymer that accelerates the binding of the specific binding partner to [25(OH)D]; and b) determining the concentration of [25(OH)D] in the sample with a single reaction mixture, wherein the vitamin D binding proteins are not removed from the sample, and no phase separation or washing steps are involved.

In some embodiments, the water miscible organic solvent is dimethyl sulfoxide, dimethyl formamide, acetonitrile, methanol, ethanol or isopropyl alcohol. The water miscible organic solvent in the final reaction mixture ranges from about 1.0 to about 40% (V/V).

In some embodiments, the binding partner is an antibody or a set of antibodies that specifically that binds [25(OH)D].

In some embodiments, the water soluble polymer is polyoxyethylene with a molecular weight from about 3,000 to about 500,000. In other embodiments, the water soluble polymer is polyvinyl pyrrolidone with a molecular weight from about 20,000 to about 750,000. In still other embodiments, the water soluble polymer is dextran with a molecular weight from about 5,000 to about 700,000. The concentration of the water soluble polymer in the final reaction mixture ranges from about 0.01 to about 5% (W/V).

In some embodiments, the sample is biological fluids including but not limited to whole blood, plasma, serum, and urine.

In some embodiments, the concentration of [25(OH)D] includes the total concentration of [25(OH)D] including [25(OH)D3] and [25(OH)D2].

In some embodiments, the assay format of the homogenous method of [25(OH)D] determination is the particle-enhanced immunoturbidimetric methods. In other embodiments, the assay format of the homogenous method of [25(OH)D] determination is the particle-enhanced immunonephelometric methods. In still other embodiments, the assay format of the homogenous method of [25(OH)D] determination is the magnetic particle (beads) based immunoassay methods.

In some embodiments, the assay format of the heterogeneous method of [25(OH)D] determination is an enzyme-linked immunosorbent assay (ELISA) method.

Any suitable antibodies can be used. In some embodiments, the antibody is polyclonal. In other embodiments, the antibody is monoclonal.

Any suitable particles can be used. In some embodiments, the particle is a nano particle including but not limited to particles made of polystyrene, polymethyl methacrylate, polymethyl naphthalene, carbon 60, co-polymer of styrene, acrylic acid, naphathalene, magnetic beads, gold, silver, silica, silicon dioxide, chromium dioxide, and titanium dioxide.

In some embodiments, the present invention provides a kit for determining 25(OH)D in a sample, the kit comprises 3 reagents including (1) a 25(OH)D dissociation solution containing a water miscible organic solvent and a water soluble polymer; (2) a 25(OH)D binding partner composition or solution containing a specific antibody against 25(OH)D; and (3) a reagent containing 25(OH)D coated particles. In an exemplary assay, samples such as plasma or serum are mixed with the dissociation solution and binding partner composition or solution, and incubated for a short period of time before addition of the third reagent containing the 25(OH)D coated particles. The 25(OH)D concentration in the sample is quantified by measuring the optical change of the reaction mixture and using a set of 25(OH)D calibrators.

In some embodiments, the present invention provides a kit for determining 25(OH)D in a sample, the kit comprises 2 reagents including (1) a 25(OH)D dissociation solution containing a water miscible organic solvent, a water soluble polymer, and an antibody against 25(OH)D; and (2) a reagent containing 25(OH)D coated particles. In an exemplary assay, samples such as plasma or serum are mixed with the dissociation solution and incubated for a short period of time before addition of the second reagent containing the 25(OH)D coated particles. The 25(OH)D concentration in the sample is quantified by measuring the optical change of the reaction mixture and using a set of 25(OH)D calibrators.

In some embodiments, the present invention provides a kit for determining 25(OH)D in a sample, the kit contains a dissociation solution comprising a water miscible organic solvent and a water soluble polymer. This kit can be used in the ELISA format with microtiter plates for 25(OH)D determination.

In some embodiments, the exemplary vitamin D assays use a water miscible organic solvent, a non-ionic polymer and nano particles. The exemplary vitamin D assays are conducted at neutral pH, in a single reaction mixture and homogenous format, and without removing any component from the samples during the assay procedure.

The present invention is further illustrated by the following exemplary embodiments.

1. A method for assaying a vitamin D moiety in a sample, which method comprises: a) contacting a sample containing or suspected of containing a vitamin D moiety with a water miscible organic solvent, a specific binding partner that specifically binds to said vitamin D moiety, if present in said sample, said binding partner being different from a natural vitamin D binding protein for said vitamin D moiety, and a water soluble polymer that facilitates binding between said specific binding partner and said vitamin D moiety; and b) assessing binding between said specific binding partner and said vitamin D moiety to determine the presence, absence and/or amount of said vitamin D moiety in said sample.

2. The method of embodiment 1, which does not comprise a step of removing the natural vitamin D binding protein for the vitamin D moiety prior to assessing binding between the specific binding partner and the vitamin D moiety.

3. The method of embodiment 1 or 2, which does not comprise a wash step.

4. The method of any of embodiments 1-3, which is conducted as a homogeneous assay.

5. The method of any of embodiments 1-4, wherein the sample is contacted with a single reaction mixture comprising the water miscible organic solvent, the specific binding partner and the water soluble polymer.

6. The method of any of embodiments 1-5, which does not comprise a step of contacting the sample with 8-anilino-1-napthalenesulfonic acid ammonium salt and/or 3-(acetonylbenzyl)-4-hydroxycoumarin.

7. The method of any of embodiments 1-5, which does not comprise a step of contacting the sample with a non-competitive displacement agent that separates the vitamin D moiety from its binding protein in the sample.

8. The method of any of embodiments 1-7, wherein the pH in a final reaction mixture comprising the sample, the water miscible organic solvent, the specific binding partner and the water soluble polymer is at 4 or higher.

9. The method of any of embodiments 1-8, wherein the pH in a final reaction mixture comprising the sample, the water miscible organic solvent, the specific binding partner and the water soluble polymer is at 13 or lower.

10. The method of any of embodiments 1-9, which does not comprise a step of contacting the sample with a vitamin D releasing composition comprising a cyclodextrin, a sodium salicylate and NaOH.

11. The method of any of embodiments 1-10, which does not comprise a step of contacting the sample with a perfluoro alkyl acid, or a salt thereof, to release 25(OH) vitamin D from vitamin D binding protein.

12. The method of any of embodiments 1-11, which does not comprise a step of contacting the sample with a serine protease with endo- and exoproteolytic activity to digest vitamin D binding proteins in the sample.

13. The method of any of embodiments 1-12, which is used to assess status of the vitamin D moiety in a subject, and the sample is a biological sample obtained and/or derived from the subject.

14. The method of embodiment 13, wherein the subject is a mammal.

15. The method of embodiment 14, wherein the mammal is a human.

16. The method of any of embodiments 13-15, wherein the sample is a biological fluid.

17. The method of embodiment 16, wherein the biological fluid is selected from the group consisting of whole blood, plasma, serum and urine.

18. The method of any of embodiments 1-17, wherein the vitamin D moiety is vitamin D₃, vitamin D₂, a vitamin D metabolite or 1,25-dihydroxyvitamin D₃ (1,25-(OH)₂D₃)

19. The method of embodiment 18, wherein the vitamin D metabolite is 25-hydroxy-vitamin D (25(OH)D).

20. The method of embodiment 19, wherein the 25(OH)D is 25(OH)D3.

21. The method of embodiment 19, wherein the 25(OH)D is 25(OH)D2.

22. The method of embodiment 19, wherein the 25(OH)D is a sum of 25(OH)D2 and 25(OH)D3.

23. The method of any of embodiments 1-22, wherein the water miscible organic solvent is selected from the group consisting of dimethyl sulfoxide, dimethyl formamide, acetonitrile, methanol, ethanol, isopropyl alcohol, dimethyl acetamide, and a combination thereof.

24. The method of embodiment 23, wherein the concentration of the water miscible organic solvent(s) in the final reaction mixture ranges from about 0.25% (V/V) to about 20% (V/V).

25. The method of any of embodiments 1-24, wherein the specific binding partner that specifically binds to the vitamin D moiety is an antibody that specifically binds to the vitamin D moiety.

26. The method of embodiment 25, wherein the antibody specifically binds to 25(OH)D.

27. The method of embodiment 25 or 26, wherein the antibody is a polyclonal antibody.

28. The method of embodiment 25 or 26, wherein the antibody is a monoclonal antibody.

29. The method of any of embodiments 1-28, wherein the water soluble polymer is a synthetic water soluble polymer and/or a natural water soluble polymer.

30. The method of embodiment 29, wherein the synthetic water soluble polymer is selected from the group consisting of a polyoxyethylene (POE), a polyvinyl pyrrolidone (PVP), a polyvinyl alcohol (PVA), a polyacrylic acid (PAA), a polyacrylamide, a poly-N-(2-hydroxypropyl)methacrylamide (HPMA), a poly-divinyl ether-maleic anhydride (DIVEMA), a polyoxazoline, a polyphosphate, a polyphosphazene, a polyethyleneimine, a polyglycosylethyl methacrylate, and a combination thereof.

31. The method of embodiment 29, wherein the natural water soluble polymer is selected from the group consisting of a chitosan derivative, a dextran, a carrageenan, a cellulose ether, a hyaluronic acid (HA), and a starch or starch based derivative, and a combination thereof.

32. The method of any of embodiments 1-28, wherein the water soluble polymer is a non-ionic water soluble polymer.

33. The method of any of embodiments 1-32, wherein the water soluble polymer has a molecular weight from about 1,000 to about 2,000,000, preferably from about 2,000 to about 1,000,000.

34. The method of any of embodiments 1-33, wherein the water soluble polymer is selected from the group consisting of polyvinyl pyrrolidone, polyethyleneimine, polyoxyethylene, and polyglycosylethyl methacrylate.

35. The method of any of embodiments 1-34, wherein the water soluble polymer is polyoxyethylene with a molecular weight from about 3,000 to about 500,000.

36. The method of embodiment 35, wherein the polyoxyethylene has a molecular weight from about 3,000, 4,000, 5,000, 6,000, 7,000, 8,000, 9,000, 10,000, 50,000, 100,000, 150,000, 200,000, 250,000, 300,000, 350,000, 400,000, 450,000, or 500,000.

37. The method of any of embodiments 1-34, wherein the water soluble polymer is polyvinyl pyrrolidone with a molecular weight from about 20,000 to about 750,000.

38. The method of embodiment 37, wherein the polyvinyl pyrrolidone has a molecular weight from about 20,000, 30,000, 40,000, 50,000, 60,000, 70,000, 80,000, 90,000, 100,000, 150,000, 200,000, 250,000, 300,000, 350,000, 400,000, 450,000, 500,000, 550,000, 600,000, 650,000, 700,000 or 750,000.

39. The method of any of embodiments 1-34, wherein the water soluble polymer is dextran with a molecular weight from about 5,000 to about 700,000.

40. The method of embodiment 39, wherein the dextran has a molecular weight from about 5,000, 6,000, 7,000, 8,000, 9,000, 10,000, 50,000, 100,000, 150,000, 200,000, 250,000, 300,000, 350,000, 400,000, 450,000, 500,000, 550,000, 600,000, 650,000 or 700,000.

41. The method of any of embodiments 1-40, wherein the concentration of the water soluble polymer in the final reaction mixture ranges from about 0.025% (W/V) to about 10% (W/V).

42. The method of any of embodiments 1-41, which is conducted using a particle-enhanced immunoturbidimetric method.

43. The method of any of embodiments 1-41, which is conducted using a particle-enhanced immunonephelometric method.

44. The method of any of embodiments 1-41, which is conducted using a magnetic particle (bead) based immunoassay method.

45. The method of any of embodiments 42-44, wherein the particle comprises polystyrene, polymethyl methacrylate, polymethyl naphthalene, poly(divinylbenzene), polyvinyl naphthalene, co-polymer of styrene, acrylic acid divinylbenzene, naphathalene, carbon 60, magnetic beads, gold, silver, silica, silicon dioxide, chromium dioxide, and/or titanium dioxide.

46. The method of any of embodiments 42-45, wherein the particle is a nanoparticle.

47. The method of embodiment 46, wherein the nanoparticle has a diameter ranging from about 30 nm to about 500 nm.

48. The method of any of embodiments 1-47, which is conducted using a homogeneous or a heterogeneous assay format.

49. The method of any of embodiments 1-48, which is conducted using a sandwich or competitive assay format.

50. The method of any of embodiments 1-49, which is conducted using a format selected from the group consisting of an enzyme-linked immunosorbent assay (ELISA), cloned enzyme donor immunoassay (CEDIA), enzyme multiplied immunoassay technique (EMIT), immunoblotting, immunoprecipitation, radioimmunoassay (RIA), immunostaining, latex agglutination, indirect hemagglutination assay (IHA), complement fixation, indirect immunofluorescent assay (IFA), nephelometry, chemiluminescence assay, lateral flow immunoassay, immunoinhibition assay and immune-avidity assay.

51. The method of any of embodiments 1-49, which is a homogenous assay conducted in a single reaction mixture without phase separation or washing step.

52. The method of any of embodiments 1-51, which has a total assay time that is about 30 minutes or shorter.

53. The method of any of embodiments 1-52, which is conducted on a general chemistry analyzer or a clinical chemistry analyzer.

54. The method of embodiment 53, wherein the general chemistry analyzer or clinical chemistry analyzer is from Roche, Hitachi, Modular P, Cobas series, Beckman/Olympus AU series, Beckman Synchron and DXC series, or Abbot Architect series.

55. A kit for assaying a vitamin D moiety in a sample, which kit comprises:

a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

56. The kit of embodiment 55, which further comprises means for assessing binding between the specific binding partner and the vitamin D moiety to determine the presence, absence and/or amount of the vitamin D moiety in the sample.

57. The kit of embodiment 55, which comprises reagents: (1) a first assay reagent comprising the water miscible organic solvent and the water soluble polymer; (2) a second assay reagent comprising a specific antibody against 25(OH)D; and (3) a third assay reagent comprising 25(OH)D coated particles.

58. The kit of embodiment 55, which comprises reagents: (1) a first assay reagent comprising the water miscible organic solvent, the water soluble polymer and a specific antibody against 25(OH)D; and (2) a second assay reagent comprising 25(OH)D coated particles.

59. The kit of embodiment 55, which comprises: (1) a first assay reagent comprising the water miscible organic solvent and the water soluble polymer, and (2) a solid surface comprising an immobilized substance that is the specific binding partner that specifically binds to a vitamin D moiety or its analog.

60. A method for assaying a vitamin D moiety in a sample using the kit of embodiment 57, which method comprises: a) forming a mixture of a sample, the first assay reagent and the second assay reagent and incubating the mixture for a period of time before adding the third assay reagent to the mixture; and b) quantifying the amount of 25(OH)D in the sample by measuring the optical change of the reaction mixture and using a set of 25(OH)D calibrators.

61. A method for assaying a vitamin D moiety in a sample using the kit of embodiment 58, which method comprises: a) forming a mixture of a sample and the first assay reagent and incubating the mixture for a period of time before adding the second assay reagent to the mixture; and b) quantifying the amount of 25(OH)D in the sample by measuring the optical change of the reaction mixture and using a set of 25(OH)D calibrators.

62. A method for assaying a vitamin D moiety in a sample using the kit of embodiment 59, which method comprises: a) forming a mixture of a sample and the first assay reagent and incubating the mixture for a period of time; and b) contacting the mixture with the solid surface to determine the presence, absence and/or amount of the vitamin D moiety in the sample.

63. A reaction mixture for assaying a vitamin D moiety in a sample, which reaction mixture comprises: a) a water miscible organic solvent; b) a specific binding partner that specifically binds to said vitamin D moiety, if present in said sample, said binding partner being different from a natural vitamin D binding protein for said vitamin D moiety; and c) a water soluble polymer that facilitates binding between said specific binding partner and said vitamin D moiety.

64. The reaction mixture of embodiment 63, which further comprises a vitamin D moiety that is bound to the specific binding partner.

65. The reaction mixture of embodiment 63 or 64, which is in a single phase.

### EXAMPLES

### Example 1

### Total 25(OH)D Assay Kit (Three Reagent Format)

The three Reagents used in this example are listed below:

### Reagent 1

0.4M Acetate buffer

### 0.1M Sodium Chloride

0.5M Ethylenediaminetetraacetic acid

### 0.0138 M Sodium Azide

20% (v/v) Dimethyl Sulfoxide
2% Poly (ethylene oxide) (Avg. mol. Wt. 100,000)
pH adjusted to 4.0

### Reagent 2

### 0.1M Tris

### 0.600 M Sodium Chloride

### 0.0138 M Sodium Azide

10 mg/L a monoclonal anti-vitamin D antibody
pH adjusted to 7.5

### Reagent 3

### 0.2 M Tris

0.1% (v/v) Tween 20
0.35% (w/v) Vitamin D-coated particles

### 0.0138 M Sodium Azide

pH adjusted to 8.0

### Assay Parameters on Hitachi 917 for 3 Reagent Format

Thirty (30) μL of sample was mixed with 150 μL of Reagent 1 inside a cuvette and incubated at 37° C. from cycle #1-5 (1.8 minutes). Fifty (50) μL of Reagent 2 was added to the mixture and incubated from cycle #6-16 (3 minutes). Fifty (50) μL of Reagent 3 was added to the mixture and incubated from cycle #17-34 (5.4 minutes). The change in absorbance at 570 nm was measured as “Endpoint” between cycle 19 and 34. Each cycle is 18 seconds in duration.

### Total 25(OH)D Assay Kit (Three Reagent Format) Performance Summary

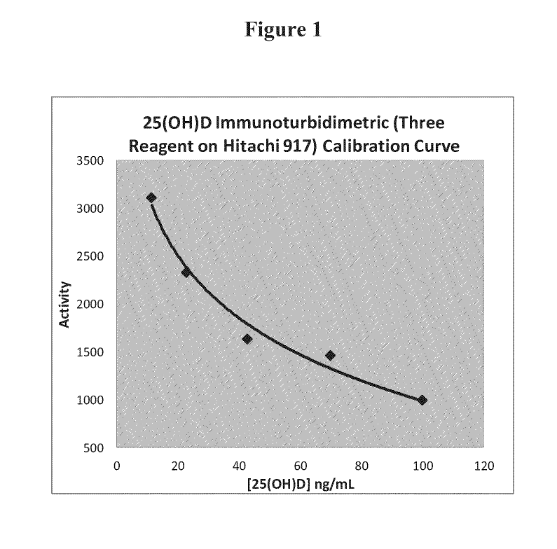

The 25(OH)D Assay kit was calibrated with serum based calibrators of known 25(OH)D concentrations. The calibration curve is shown in FIG. 1.

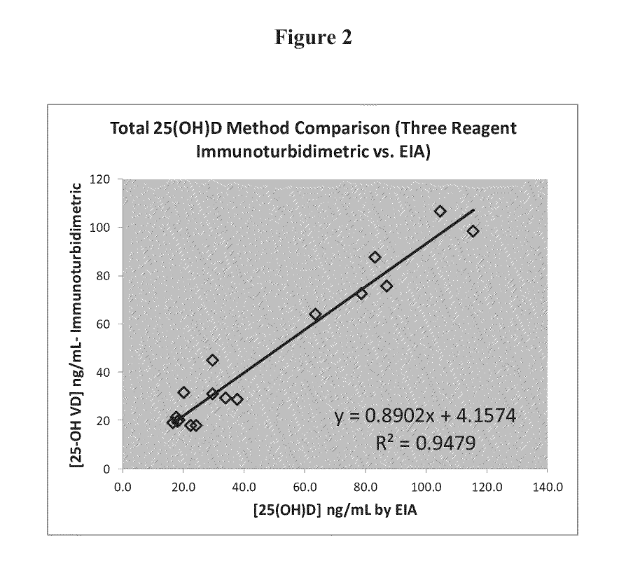

After successful calibration of the 25(OH)D assay kit, patient serum samples were tested and the results were compared to a commercially available enzyme immunosorbent assay (EIA) for total vitamin D manufactured by IDS, U.K. The results are shown in Table 1 and FIG. 2.

TABLE 1

| | | [25-OH vitamin D] ng/mL by |
|---|---|---|
| | [25-OH vitamin D] ng/mL | Immunoturbidimetric |
| Sample | by EIA | on Hitachi 917 |
| Serum 1 | 22.5 | 17.8 |
| Serum 2 | 17.6 | 21.2 |
| Serum 3 | 115.6 | 98.5 |
| Serum 4 | 20.2 | 31.4 |
| Serum 5 | 78.8 | 72.4 |
| Serum 6 | 63.6 | 63.8 |
| Serum 7 | 104.8 | 106.6 |
| Serum 8 | 87 | 75.5 |
| Serum 9 | 33.9 | 29.3 |
| Serum 10 | 24.3 | 17.8 |
| Serum 11 | 16.6 | 19 |
| Serum 12 | 18.1 | 19.7 |
| Serum 13 | 83.3 | 87.5 |
| Serum 14 | 29.6 | 44.9 |
| Serum 15 | 18.6 | 20 |
| Serum 16 | 37.8 | 28.5 |
| Serum 17 | 29.7 | 31 |

### Example 2

### Total 25(OH)D Assay Kit (Two Reagent Format)

The two Reagents used in this example are listed below:

### Reagent 1

### 0.5M MES

### 0.1M Sodium Chloride

0.05 M Ethylenediaminetetraacetic acid

### 0.0138 M Sodium Azide

14% (v/v) Dimethyl Sulfoxide
1% Poly (ethylene oxide) (Avg. mol. Wt. 100,000)
5.0 mg/L a monoclonal anti-Vitamin D antibody
pH adjusted to 5.3

### Reagent 2

### 0.25 M Tris

0.1% (v/v) Tween 20
0.25% (w/v) Vitamin D-coated particles (same as in Example 1)

### 0.0138 M Sodium Azide

pH adjusted to 8.0

### Assay Parameters on Modular P for Two Reagent Format

Thirty (30) μL of sample was mixed with 200 μL of Reagent 1 inside a cuvette and incubated at 37° C. from cycle #1-16 (4.8 minutes). Fifty (50) μL of Reagent 2 was added to the mixture and incubated from cycle #17-34 (5.4 minutes). The change in absorbance at 570 nm was measured as “Endpoint” between cycle #19 and 34. Each cycle is 18 seconds in duration.

### Total 25(OH)D Assay Kit (Two Reagent Format) Performance Summary

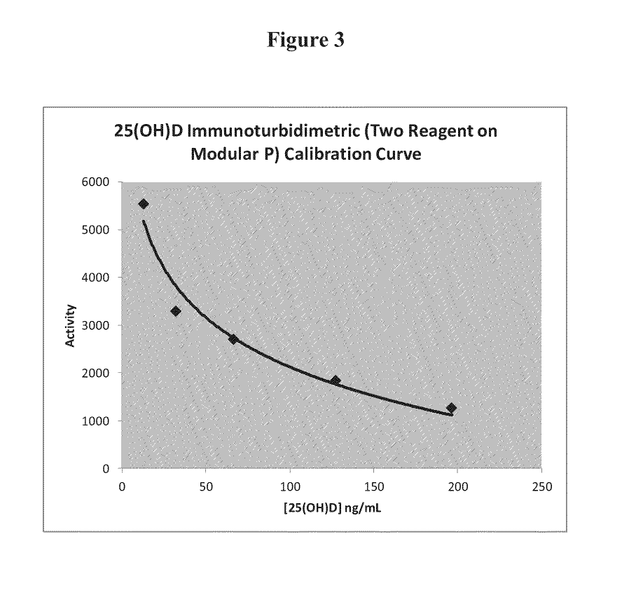

The 25(OH)D Assay kit was calibrated with serum based calibrators of known 25(OH)D concentrations. The calibration curve is shown in FIG. 3.

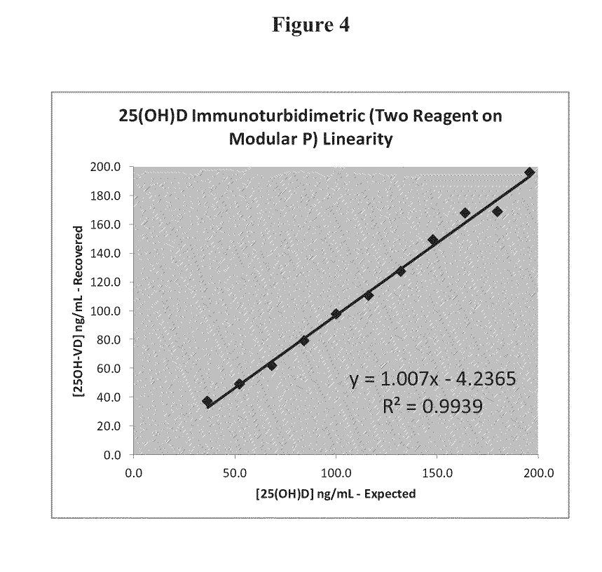

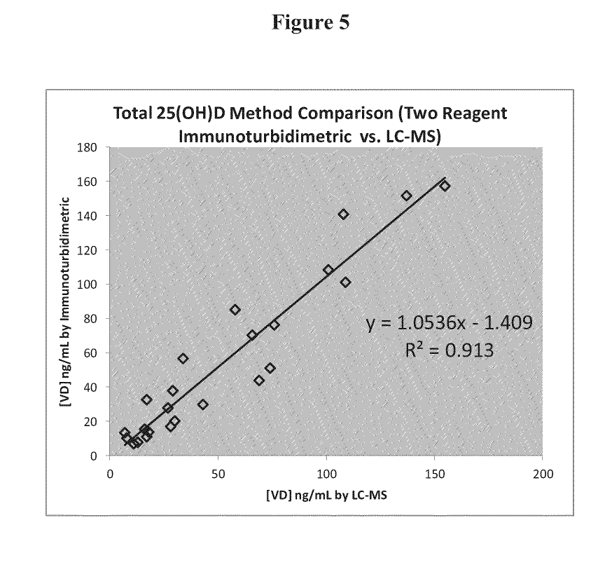

After successful calibration of the 25(OH)D assay kit, controls and linearity standards were tested. In addition, patient serum samples were tested and the results were compared to a liquid chromatography-mass spectrophotometric (LC-MS) method for total vitamin D. The results are shown below (Tables 2-4 and FIGS. 4 and 5).

TABLE 2
Imprecision

| | Control 1 | Control 2 | Control 3 |
|---|---|---|---|
| [25(OH)D] ng/mL Mean (n = 10) | 20.9 | 32.12 | 73.01 |
| Std Dev. | 0.82 | 1.75 | 2.43 |
| % CV | 3.9% | 5.5% | 3.3% |

TABLE 3
Linearity

| | | [25(OH)D] ng/mL |
|---|---|---|
| | [25(OH)D] ng/mL | Recovered |
| Level | Expected | (Mean of 3 replicates) |
| 1 | 36.6 | 36.6 |
| 2 | 52.5 | 48.6 |
| 3 | 68.5 | 61.7 |
| 4 | 84.4 | 78.7 |
| 5 | 100.3 | 97.2 |
| 6 | 116.3 | 110.4 |
| 7 | 132.2 | 126.9 |
| 8 | 148.1 | 149.1 |
| 9 | 164.0 | 167.4 |
| 10 | 180.0 | 168.7 |
| 11 | 195.9 | 195.9 |

TABLE 4
Method Comparison

| Serum | [25(OH)D] ng/mL by | [25(OH)D] ng/mL by |
|---|---|---|
| Sample | LCMS | Immunoturbidimetric |
| 1B | 18 | 13.6 |
| 2B | 16 | 15.4 |
| 3B | 17 | 10.9 |
| 4B | 27 | 27.7 |
| 5B | 28 | 16.9 |
| 6B | 13 | 7.7 |
| 7B | 11 | 6.8 |
| 8B | 17 | 32.4 |
| 9B | 7 | 13.5 |
| 10B | 8 | 10.2 |
| 21B | 74 | 50.8 |
| 22B | 43 | 29.6 |
| 23B | 108 | 140.7 |
| 24B | 155 | 157.3 |
| 25B | 76 | 76.3 |
| 26B | 109 | 100.9 |
| 27B | 34 | 56.5 |
| 28B | 29 | 37.8 |
| 29B | 30 | 20.2 |
| 30B | 137 | 151.5 |
| 31B | 58 | 85.2 |
| 32B | 101 | 108.4 |
| 33B | 66 | 70.2 |
| 35B | 69 | 43.8 |

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for assaying a vitamin D moiety in a sample, which method comprises:
a) contacting a sample containing or suspected of containing a vitamin D moiety with a water miscible organic solvent, a specific binding partner that specifically binds to said vitamin D moiety, if present in said sample, said binding partner being different from a natural vitamin D binding protein for said vitamin D moiety, and a water soluble polymer that facilitates binding between said specific binding partner and said vitamin D moiety; and
b) assessing binding between said specific binding partner and said vitamin D moiety to determine the presence, absence and/or amount of said vitamin D moiety in said sample.

2. The method of claim 1, which does not comprise a step of removing the natural vitamin D binding protein for the vitamin D moiety prior to assessing binding between the specific binding partner and the vitamin D moiety, and/or does not comprise a wash step.

3. The method of claim 1, which is conducted as a homogeneous assay.

4. The method of claim 1, wherein the sample is contacted with a single reaction mixture comprising the water miscible organic solvent, the specific binding partner and the water soluble polymer.

5. The method of claim 1, wherein the pH in a final reaction mixture comprising the sample, the water miscible organic solvent, the specific binding partner and the water soluble polymer ranges from about 4 to about 13.

6. The method of claim 1, which is used to assess status of the vitamin D moiety in a subject, and the sample is a biological sample obtained and/or derived from the subject.

7. The method of claim 1, wherein the vitamin D moiety is 25(OH)D2, 25(OH)D 3 or a sum of 25(OH)D2 and 25(OH)D3.

8. The method of claim 1, wherein the water miscible organic solvent is selected from the group consisting of dimethyl sulfoxide, dimethyl formamide, acetonitrile, methanol, ethanol, isopropyl alcohol, dimethyl acetamide, and a combination thereof.

9. The method of claim 8, wherein the concentration of the water miscible organic solvent(s) in the final reaction mixture ranges from about 0.25% (V/V) to about 20% (V/V).

10. The method of claim 1, wherein the specific binding partner that specifically binds to the vitamin D moiety is an antibody that specifically binds to the vitamin D moiety.

11. The method of claim 10, wherein the antibody specifically binds to 25(OH)D.

12. The method of claim 1, wherein the water soluble polymer is a synthetic water soluble polymer and/or a natural water soluble polymer.

13. The method of claim 12, wherein the synthetic water soluble polymer is selected from the group consisting of a polyoxyethylene (POE), a polyvinyl pyrrolidone (PVP), a polyvinyl alcohol (PVA), a polyacrylic acid (PAA), a polyacrylamide, a poly-N-(2-hydroxypropyl)methacrylamide (HPMA), a poly-divinyl ether-maleic anhydride (DIVEMA), a polyoxazoline, a polyphosphate, a polyphosphazene, a polyethyleneimine, a polyglycosylethyl methacrylate, and a combination thereof, and the natural water soluble polymer is selected from the group consisting of a chitosan derivative, a dextran, a carrageenan, a cellulose ether, a hyaluronic acid (HA), and a starch or starch based derivative, and a combination thereof.

14. The method of claim 1, wherein the water soluble polymer is a non-ionic water soluble polymer.

15. The method of claim 1, wherein the water soluble polymer has a molecular weight from about 1,000 to about 2,000,000.

16. The method of claim 1, wherein the concentration of the water soluble polymer in the final reaction mixture ranges from about 0.025% (W/V) to about 10% (W/V).

17. The method of claim 1, which is conducted using a particle-enhanced immunoturbidimetric method, a particle-enhanced immunonephelometric method or a magnetic particle (bead) based immunoassay method.

18. The method of claim 17, wherein the particle comprises polystyrene, polymethyl methacrylate, polymethyl naphthalene, poly(divinylbenzene), polyvinyl naphthalene, co-polymer of styrene, acrylic acid divinylbenzene, naphathalene, carbon 60, magnetic beads, gold, silver, silica, silicon dioxide, chromium dioxide, and/or titanium dioxide, or the particle is a nanoparticle.

19. The method of claim 1, which is a homogenous assay conducted in a single reaction mixture without phase separation or washing step.

20. The method of claim 1, which has a total assay time that is about 30 minutes or shorter.

21. The method of claim 1, which is conducted on a general chemistry analyzer or a clinical chemistry analyzer.

22. A kit for assaying a vitamin D moiety in a sample, which kit comprises:
a) a water miscible organic solvent;
b) a specific binding partner that specifically binds to said vitamin D moiety, if present in said sample, said binding partner being different from a natural vitamin D binding protein for said vitamin D moiety; and
c) a water soluble polymer that facilitates binding between said specific binding partner and said vitamin D moiety.

23. The kit of claim 22, which further comprises means for assessing binding between the specific binding partner and the vitamin D moiety to determine the presence, absence and/or amount of the vitamin D moiety in the sample.

24. The kit of claim 22, which comprises reagents:
(1) a first assay reagent comprising the water miscible organic solvent and the water soluble polymer;
(2) a second assay reagent comprising a specific antibody against 25(OH)D; and
(3) a third assay reagent comprising 25(OH)D coated particles.

25. The kit of claim 22, which comprises reagents:
(1) a first assay reagent comprising the water miscible organic solvent, the water soluble polymer and a specific antibody against 25(OH)D; and
(2) a second assay reagent comprising 25(OH)D coated particles.

26. The kit of claim 22, which comprises:
(1) a first assay reagent comprising the water miscible organic solvent and the water soluble polymer, and
(2) a solid surface comprising an immobilized substance that is the specific binding partner that specifically binds to a vitamin D moiety or its analog.

27. A method for assaying a vitamin D moiety in a sample using the kit of claim 24, which method comprises:
a) forming a mixture of a sample, the first assay reagent and the second assay reagent and incubating the mixture for a period of time before adding the third assay reagent to the mixture; and
b) quantifying the amount of 25(OH)D in the sample by measuring the optical change of the reaction mixture and using a set of 25(OH)D calibrators.

28. A method for assaying a vitamin D moiety in a sample using the kit of claim 25, which method comprises:
a) forming a mixture of a sample and the first assay reagent and incubating the mixture for a period of time before adding the second assay reagent to the mixture; and
b) quantifying the amount of 25(OH)D in the sample by measuring the optical change of the reaction mixture and using a set of 25(OH)D calibrators.

29. A method for assaying a vitamin D moiety in a sample using the kit of claim 26, which method comprises:
a) forming a mixture of a sample and the first assay reagent and incubating the mixture for a period of time; and
b) contacting the mixture with the solid surface to determine the presence, absence and/or amount of the vitamin D moiety in the sample.

30. A reaction mixture for assaying a vitamin D moiety in a sample, which reaction mixture comprises:
a) a water miscible organic solvent;
b) a specific binding partner that specifically binds to said vitamin D moiety, if present in said sample, said binding partner being different from a natural vitamin D binding protein for said vitamin D moiety; and
c) a water soluble polymer that facilitates binding between said specific binding partner and said vitamin D moiety.
